(19) United States
(12) Patent Application Publication
(10) Pub. No.: US 2009/0293429 A1
(43) Pub. Date: Dec. 3, 2009

(54) BEVERAGE BOTTLING PLANT WITH METHOD AND APPARATUS FOR CLEANING, FILLING, AND CLOSING BOTTLES

(76) Inventor: Volker TILL, Hofheim am Taunus (DE)

Correspondence Address: NILS H. LJUNGMAN & ASSOCIATES, P. O. BOX 130, GREENSBURG, PA, 15601-0130, US

(21) Appl. No.: 12/326,484

(22) Filed: Dec. 2, 2008

Related U.S. Application Data
(63) Continuation-in-part of application No. PCT/EP2007/004599, filed on May 24, 2007.

(30) Foreign Application Priority Data
Jun. 2, 2006 (DE) 10 2006 026 278.6

(51) Int. Cl. B65B 55/02

(52) U.S. Cl. 53/425; 53/167

(57) ABSTRACT

A beverage bottling plant with method and apparatus for cleaning, filling, and closing bottles. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/EP2007/004599, filed on May 24, 2007, which claims priority from Federal Republic of Germany Patent Application No. 10 2006 026 278.6, filed on Jun. 2, 2006. International Patent Application No. PCT/EP2007/004599 was pending as of the filing date of this application. The United States was an elected state in International Patent Application No. PCT/EP2007/004599.

### BACKGROUND

1. Technical Field

The present application relates to a beverage bottling plant with a method and apparatus for treating, filling, and closing bottles or similar containers.

2. Background Information

Background information is for informational purposes only and does not necessarily admit that subsequently mentioned information and publications are prior art.

A beverage bottling plant for filling bottles with a liquid beverage filling material can possibly comprise a beverage filling machine, which is often a rotary filling machine, with a plurality of beverage filling positions, each beverage filling position having a beverage filling device for filling bottles with liquid beverage filling material. The filling devices may have an apparatus designed to introduce a predetermined volume of liquid beverage filling material into the interior of bottles to a substantially predetermined level of liquid beverage filling material.

Some beverage bottling plants may possibly comprise filling arrangements that receive a liquid beverage material from a toroidal or annular vessel, in which a supply of liquid beverage material is stored. The toroidal vessel may also be connected to at least one external reservoir or supply of liquid beverage material by a conduit or supply line. In some circumstances it may even be possible that a beverage bottling plant has two external supply reservoirs, each of which may be configured to store either the same liquid beverage product or different products. These reservoirs could possibly be connected to the toroidal or annular vessel by corresponding supply lines, conduits, or other arrangements. It is also possible that the external supply reservoirs could be in the form of simple storage tanks, or in the form of liquid beverage product mixers.

A wide variety of types of filling elements are used in filling machines in beverage bottling or container filling plants for dispensing a liquid product into bottles, cans or similar containers, including but not limited to filling processes that are carried out under counterpressure for the bottling of carbonated beverages. The apparatus designed to introduce a predetermined flow of liquid beverage filling material further comprises an apparatus that is designed to terminate the filling of the beverage bottles upon the liquid beverage filling material reaching the predetermined level in bottles. There may also be provided a conveyer arrangement that is designed to move bottles, for example, from an inspecting machine to the filling machine.

After a filling process has been completed, the filled beverage bottles are transported or conveyed to a closing machine, which is often a rotary closing machine. A revolving or rotary machine comprises a rotor, which revolves around a central, vertical machine axis. There may further be provided a conveyer arrangement configured to transfer filled bottles from the filling machine to the closing station. A transporting or conveying arrangement can utilize transport star wheels as well as linear conveyors. A closing machine closes bottles by applying a closure, such as a screw-top cap or a bottle cork, to a corresponding bottle mouth. Closed bottles are then usually conveyed to an information adding arrangement, wherein information, such as a product name or a manufacturer's information or logo, is applied to a bottle. A closing station and information adding arrangement may be connected by a corresponding conveyer arrangement. Bottles are then sorted and packaged for shipment out of the plant.

Many beverage bottling plants may also possibly comprise a rinsing arrangement or rinsing station to which new, non-return and/or even return bottles are fed, prior to being filled, by a conveyer arrangement, which can be a linear conveyor or a combination of a linear conveyor and a starwheel. Downstream of the rinsing arrangement or rinsing station, in the direction of travel, rinsed bottles are then transported to the beverage filling machine by a second conveyer arrangement that is formed, for example, by one or more starwheels that introduce bottles into the beverage filling machine.

It is a further possibility that a beverage bottling plant for filling bottles with a liquid beverage filling material can be controlled by a central control arrangement, which could be, for example, a computerized control system that monitors and controls the operation of the various stations and mechanisms of the beverage bottling plant.

At least one possible embodiment relates to a method for the treatment of bottles, cans or similar containers by activation of a treatment medium in the respective container by the input of energy, wherein the activation is accomplished by activating the treatment medium with a source and to an apparatus for the treatment of bottles, cans or similar containers by activation of a treatment medium in the respective container by an input of energy, with at least one transport element that moves the container during the treatment on a transport line, wherein at least one activator directed at the individual container is provided for the activation of the treatment medium in the respective container.

Some treatments of bottles or similar containers, in at least one possible embodiment for sterilization with the use of a treatment or sterilization medium (referred to simply as a treatment medium below), can comprise, for example, an aqueous solution of hydrogen peroxide (H₂O₂) in a vapor and/or gaseous medium, for example in the form of an aerosol in steam and/or air.

In some methods, the treatment medium, after it is introduced into a container, may be impacted or activated by a heated gaseous medium such as hot air, for example, and possibly, among other things, to vaporize a portion of the water in the treatment medium and/or to break down the hydrogen peroxide (H₂O₂) into its active components, oxygen and water or steam (2H₂O₂=2H₂O═O₂=45 cal) as well as for the subsequent removal of the treatment medium, in at least one possible embodiment water and steam, and the decomposition products that are generated during the activation. The steps in the process are thereby performed in treatment stations which can be formed, for example, on the periphery of a rotor that is driven in rotation around a vertical machine axis. The sterilized containers can then be transported downstream to a filling machine for the sterile or aseptic bottling of a liquid.

It is increasingly common to use bottles, as well as other containers, which are manufactured by blow molding from preforms made of a thermoplastic material, for example, by stretch blow molding, in at least one possible embodiment PET bottles. Such containers made out of plastic, and in at least one possible embodiment the bottles or containers manufactured by stretch blow molding, tend to deform or to shrink when they are exposed to heat, as a result of which, among other things, the shape and volume of such containers can be modified in an undesirable manner. Other types of damage can also occur when the containers are exposed to heat, as a result of which the sterilization of such containers is problematic, and deformed or damaged containers may be transferred out of a production line and rejected, which results at the very minimum in increased costs.

For these reasons, containers that are made of plastic, in at least one possible embodiment containers that are manufactured from plastic preforms by stretch blow molding, but also other heat-sensitive containers, cannot be sterilized with the conventional treatment methods. A number of different methods have already been suggested to resolve this problem. However, these methods are relatively complex, expensive and time-consuming.

### OBJECT OR OBJECTS

The object of at least one possible embodiment of the present application is to describe a method with which a treatment of heat-sensitive containers, as well as also a treatment of plastic containers, can be performed by the activation of at least one treatment medium with the input of energy, without the risk of deformation, shrinkage or any other damage to the individual container.

### SUMMARY

At least one possible embodiment according to the present application teaches that this object can be accomplished by a method for the treatment, in one possible embodiment for the sterilization, of bottles, cans or similar containers by activation of a treatment medium in the respective container by the input of energy, wherein the activation is accomplished by activating the treatment medium with an electromagnetic radiation. An apparatus for the performance of this method is described as an apparatus for the treatment, in one possible embodiment for the sterilization, of bottles, cans or similar containers by activation of a treatment medium in the respective container by the input of energy, with at least one transport element that moves the container during the treatment on a transport line, wherein at least one generator for the generation of an electromagnetic radiation directed at the individual container is provided for the activation of the treatment medium in the respective container.

In the method according to at least one possible embodiment according to the present application, the activation and the energy input for this activation are in the form of the application of electromagnetic energy to the at least one treatment medium, i.e. by applying an electromagnetic alternating field or with electromagnetic radiation, whereby the frequency spectrum of the electromagnetic alternating field or of the electromagnetic radiation is selected so that at least one frequency is equal or approximately equal to the resonance frequency of at least one component of the at least one treatment medium.

It has been found that, surprisingly, during the activation of treatment media such as hydrogen peroxide, for example, there may be a small increase in the temperature of the treatment medium and of the container to be treated if the treatment medium is excited in its resonance frequency.

The cause of this effect is the fact that as a result of the excitation of the molecules of the treatment medium in their resonance frequency, there are elevated dynamic effects between the individual components, for example between the atoms of the molecules of the treatment medium, so that the connection of the individual atoms with one another is broken essentially by the mechanical oscillation forces that are exerted between them, instead of by the thermal oscillations that accompany a temperature increase, or as a result of the greater Bernoulli forces that occur at higher temperatures upon the collision of molecules and/or atoms.

At least one possible embodiment of the present application also teaches that the activation frequency equals a multiple and/or a fraction of the resonance frequency of the at least one treatment medium, whereby in at least one possible embodiment, it may an even-numbered integer multiple or an even-numbered integer fraction of the resonance frequency, because in this method, the energy input or the excitation takes place at a frequency and in correct phase.

With regard to the resonance frequencies, it may be recalled that most substances have one resonance frequency per degree of freedom, whereby these frequencies can also vary as a function of the aggregate condition.

The frequency of the electromagnetic radiation can therefore be, for example, in the microwave range or the range of X-rays.

As the treatment medium, a liquid and/or gaseous and/or vapor medium can be used, a medium which contains a plurality of components, such as, for example, hydrogen peroxide, e.g. in solution, and also in one possible embodiment in an aqueous solution. The corresponding treatment medium can then be, for example, an aerosol comprising a vapor (steam) and/or gas or a gas mixtures such as air, for example, and the solution, in one possible embodiment an aqueous solution of hydrogen peroxide (H₂O₂) in a concentration that is sufficient for the treatment.

As a result of the method according to at least one possible embodiment, the energy input that effects the activation takes place directly or at least essentially directly into the at least one treatment medium, the individual container is at least primarily not heated directly by the energy input, so that a treatment becomes possible which protects the container and may prevent deformation, shrinkage or any other damage to the container.

The above-discussed embodiments of the present invention will be described further hereinbelow. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

Developments of at least one possible embodiment are described according to the present application. At least one possible embodiment is explained in greater detail below on the basis of the exemplary embodiment illustrated in the accompanying figures, in which:

FIG. 1 is a simplified plan view of an apparatus for the sterilization of bottles or similar containers, such as for a subsequent cold aseptic bottling of a liquid, such as, for example, for the cold aseptic bottling of fruit juices, milk products, etc.;

FIG. 2 is a simplified illustration of a treatment head of the apparatus illustrated in FIG. 1;

FIG. 3 is a simplified illustration of system for filling containers; and

FIG. 4 is a simplified illustration of a bottle cleaning machine.

### DESCRIPTION OF EMBODIMENT OR EMBODIMENTS

The apparatus which is designated 1 in general in the accompanying figures is used for the sterilization of heat-sensitive bottles 2 by the introduction into the bottles 2 of at least one treatment medium in the form of an aerosol comprising air and/or steam and an aqueous solution of hydrogen peroxide (H₂O₂), by the activation of the treatment medium by the energy input and by the removal or suctioning of residue and/or decomposition products of the activated treatment medium.

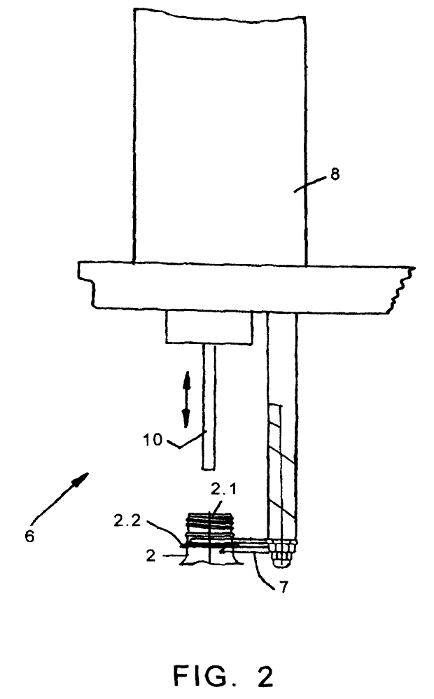

The apparatus 1, to which the bottles 2, which are made of plastic, for example PET, are delivered standing upright, i.e. with the bottle axis oriented in the vertical direction, by means of a conveyor 3 and by means of an inlet star wheel 4 which forms the bottle inlet, a rotor 5 which is driven in rotation around a vertical machine axis, whereby the rotor forms a plurality of treatment stations 6 on its periphery. These treatment stations, as illustrated in FIG. 2, have a container carrier 7 on which the bottles 2 are held during the treatment on a flange 2.2 which is provided in the vicinity of the bottle mouth 2.1, and of a treatment head 8.

The treated and/or sterilized bottles 2 are fed via a transport star wheel 9 that forms the bottle outlet or the container outlet of the apparatus 1 to an additional application, i.e. to a filling machine, for example.

Each treatment head 8 has, among other things, a treatment tube 10 which can be introduced with its lower end through the bottle mouth 2.1 into the respective bottle 2 provided in the treatment station 6, and from which treatment medium is introduced into the interior of the bottle for the treatment.

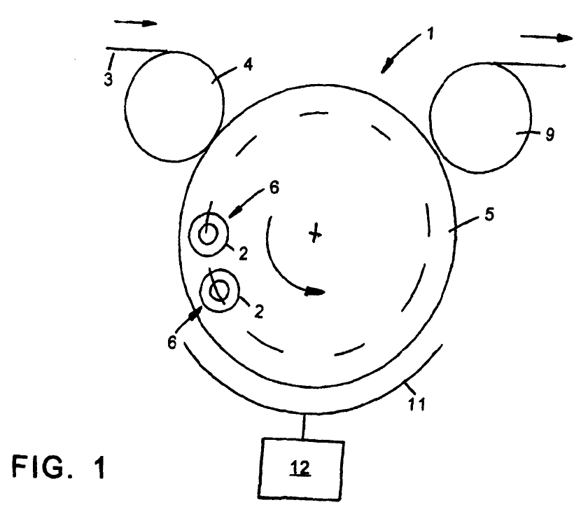

After the introduction of the treatment medium or also during the introduction of the treatment medium, the treatment station 6 in question and thus the bottle provided in it travels with the rotational movement of the rotor 5 into the vicinity of an activator which is identified schematically in FIG. 1 as 11, in which the treatment medium is activated, and in one possible embodiment by the input of energy, including among other things a decomposition of the hydrogen peroxide, resulting in the formation of water or steam and of sterilizing oxygen. In the activator 11, as a result of the energy input, there is also a maintenance of the temperature or an increase of the temperature of the steam over an angular range of the rotation of the rotor 5 which is sufficient for the sterilization of the bottles 2. However, the energy input that effects the activation is such that the temperature-sensitive or heat-sensitive bottles 2 are not damaged during this sterilization, and often are not deformed.

The activator 11 is realized so that the energy for the activation is input in the form of an electromagnetic alternating field or electromagnetic radiation (microwave radiation) which acts on the dipole molecules of at least one component of the treatment medium and thereby effects a heating of the treatment medium, without a primary heating of the bottles 2 or of the plastic material of these bottles 2. As a result, it is possible to achieve an effective activation of the treatment medium and thus an effective treatment and sterilization of the bottles 2 without causing any damage and/or deformation to the bottles 2 which are made of plastic. The activator 11 comprises, among other things, a generator 12, for example in the form of a magnetron for the generation of the electromagnetic radiation, and is located with its outlet in the path of movement of the individual treatment stations 6 so that it does not move with the rotor 5, for example also in the form of a slotted waveguide which is connected with the generator 12. It thereby also becomes possible to provide a plurality of such generators outside the rotor 5 one after the other in the direction of rotation of the rotor.

To achieve the optimum possible activation of the treatment medium and to also achieve the sterilization of these bottles in the desired quality at a high production rate of the apparatus 1, i.e. with a high number of bottles 2 treated per unit of time, the frequency spectrum of the electromagnetic alternating field used is tuned to the resonance frequency of at least one component of the treatment medium, of the component to be activated of the treatment medium (e.g., water and/or hydrogen peroxide), i.e. the frequency of the electromagnetic alternating field is equal to this resonance frequency and/or to a multiple and/or a fraction thereof.

After the activation of the treatment medium, the treatment medium and the components that are generated during the activation, such as water and steam, and during the activation of the decomposition products produced (e.g., free radicals from the decomposition of H₂O₂) are removed from the interior of the individual bottle 2, for example by blowing out the bottle with sterile air via the treatment tube 10 of the respective treatment head 8 and/or by suction, for example by means of the treatment tube 10 or by means of a suction head which is placed against or in the vicinity of the bottle mouth 2.1.

The temperature of the treatment medium when it is introduced into the individual bottle can be the same as the ambient temperature, for example. Basically, it is also possible to introduce the pre-heated treatment medium into the bottles 2, whereby this pre-heating temperature is significantly lower than a critical temperature that could lead to damage to the bottles 2 or to a deformation of the bottles 2.

In the preceding description, it was assumed that the introduction, the activation and the removal of the treatment medium and of the decomposition products formed during the activation were performed in a chronological sequence, one after the other, i.e. in adjacent angular ranges of the rotational movement of the rotor 5 between the inlet star wheel 4 and the outlet star wheel 9.

Basically, it is also possible to configure the process so that the individual treatment phases partly overlap one another or are performed during a continuous or substantially continuous feeding of the at least one treatment medium simultaneously or substantially simultaneously with its continuous or substantially continuous activation by the input of energy by means of the electromagnetic alternating field, and thereby then also of sucking out the treatment medium, for example hot water or steam, and the decomposition products that are formed during the activation.

In the preceding description, it was assumed that the treatment medium is an aqueous hydrogen peroxide solution. Other treatment media, such as for example sterile water with or without additives, can basically also be used in the method described above.

In the preceding description, it was also assumed that the energy input for the activation, i.e. for the evaporation of the water and for the decomposition of the hydrogen peroxide, is produced by an electromagnetic alternating field or by electromagnetic radiation (microwave radiation) with the use of at least one generator 12 in the form of a magnetron that generates this electromagnetic alternating field. Other methods and types of apparatus can also be used for the introduction of the energy for the activation of the treatment medium, for example for an activation by X-ray radiation.

The at least one generator, such as a magnetron for example, which generates the electromagnetic alternating field, can also be provided on the rotor 5 and rotating with the rotor, so that it can act on the treatment stations 6. It is thereby also possible to locate a plurality of generators on the rotor 5 in various positions.

In the preceding description it was assumed that the at least one treatment medium is introduced into the respective bottle. Basically, however, it is also possible to realize and/or to pre-treat the bottles so that the bottles that are delivered to the treatment stations 6 are already provided with the at least one treatment medium, for example also in the form of a treatment medium that diffuses into the wall of the respective bottle 2, so that during the rotational movement of the rotor 5 the actions that remain to be performed are the activation of the treatment medium and the removal of the activated treatment medium and of the decomposition products that are formed.

In the preceding description, it was also assumed that the bottles 2 are treated on the single rotor 5. Of course it is also possible to improve the duration and quality of the treatment by providing an additional rotor or an additional transport element on the rotor 5 in the direction of transport of the bottles 2, so that the treatment can then be distributed over a plurality of rotors or transport elements.

At least one possible embodiment was described above on the basis of exemplary embodiments. It goes without saying that numerous modifications are possible without thereby going beyond the teaching of at least one possible embodiment.

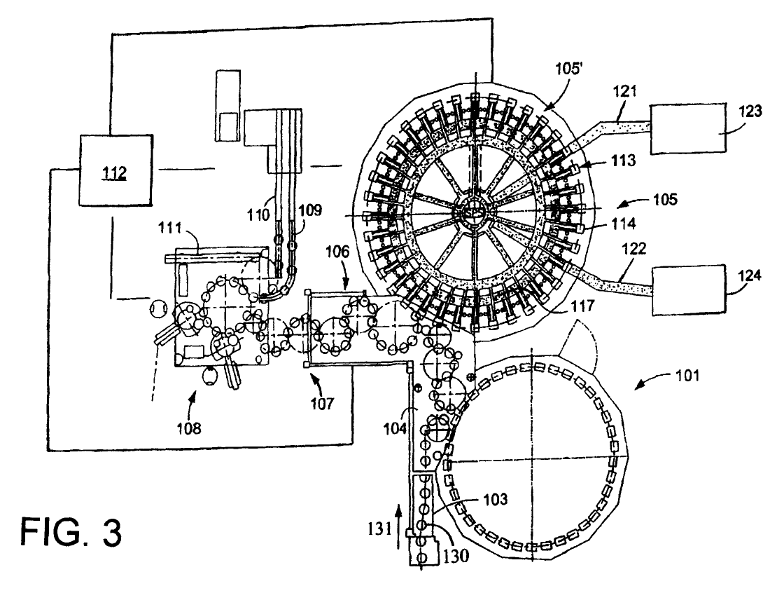

FIG. 3 shows schematically the main components of one possible embodiment example of a system for filling containers, i.e. a beverage bottling plant for filling bottles 130 with at least one liquid beverage, in accordance with at least one possible embodiment, in which system or plant could possibly be utilized at least one aspect, or several aspects, of the embodiments disclosed herein.

FIG. 3 shows a rinsing arrangement or rinsing station 101, to which the containers, namely bottles 130, are fed in the direction of travel as indicated by the arrow 131, by a first conveyer arrangement 103, which can be a linear conveyor or a combination of a linear conveyor and a starwheel. Downstream of the rinsing arrangement or rinsing station 101, in the direction of travel as indicated by the arrow 131, the rinsed bottles 130 are transported to a beverage filling machine 105 by a second conveyer arrangement 104 that is formed, for example, by one or more starwheels that introduce bottles 130 into the beverage filling machine 105.

The beverage filling machine 105 shown is of a revolving or rotary design, with a rotor 105′, which revolves around a central, vertical machine axis. The rotor 105′ is designed to receive and hold the bottles 130 for filling at a plurality of filling positions 113 located about the periphery of the rotor 105′. At each of the filling positions 103 is located a filling arrangement 114 having at least one filling device, element, apparatus, or valve. The filling arrangements 114 are designed to introduce a predetermined volume or amount of liquid beverage into the interior of the bottles 130 to a predetermined or desired level.

The filling arrangements 114 receive the liquid beverage material from a toroidal or annular vessel 117, in which a supply of liquid beverage material is stored under pressure by a gas. The toroidal vessel 117 is a component, for example, of the revolving rotor 105′. The toroidal vessel 117 can be connected by means of a rotary coupling or a coupling that permits rotation. The toroidal vessel 117 is also connected to at least one external reservoir or supply of liquid beverage material by a conduit or supply line. In the embodiment shown in FIG. 1A, there are two external supply reservoirs 123 and 124, each of which is configured to store either the same liquid beverage product or different products. These reservoirs 123, 124 are connected to the toroidal or annular vessel 117 by corresponding supply lines, conduits, or arrangements 121 and 122. The external supply reservoirs 123, 124 could be in the form of simple storage tanks, or in the form of liquid beverage product mixers, in at least one possible embodiment.

As well as the more typical filling machines having one toroidal vessel, it is possible that in at least one possible embodiment there could be a second toroidal or annular vessel which contains a second product. In this case, each filling arrangement 114 could be connected by separate connections to each of the two toroidal vessels and have two individually-controllable fluid or control valves, so that in each bottle 130, the first product or the second product can be filled by means of an appropriate control of the filling product or fluid valves.

Downstream of the beverage filling machine 105, in the direction of travel of the bottles 130, there can be a beverage bottle closing arrangement or closing station 106 which closes or caps the bottles 130. The beverage bottle closing arrangement or closing station 106 can be connected by a third conveyer arrangement 107 to a beverage bottle labeling arrangement or labeling station 108. The third conveyor arrangement may be formed, for example, by a plurality of starwheels, or may also include a linear conveyor device.

In the illustrated embodiment, the beverage bottle labeling arrangement or labeling station 108 has at least one labeling unit, device, or module, for applying labels to bottles 130. In the embodiment shown, the labeling arrangement 108 is connected by a starwheel conveyer structure to three output conveyer arrangements: a first output conveyer arrangement 109, a second output conveyer arrangement 110, and a third output conveyer arrangement 111, all of which convey filled, closed, and labeled bottles 130 to different locations.

The first output conveyer arrangement 109, in the embodiment shown, is designed to convey bottles 130 that are filled with a first type of liquid beverage supplied by, for example, the supply reservoir 123. The second output conveyer arrangement 110, in the embodiment shown, is designed to convey bottles 130 that are filled with a second type of liquid beverage supplied by, for example, the supply reservoir 124. The third output conveyer arrangement 111, in the embodiment shown, is designed to convey incorrectly labeled bottles 130. To further explain, the labeling arrangement 108 can comprise at least one beverage bottle inspection or monitoring device that inspects or monitors the location of labels on the bottles 130 to determine if the labels have been correctly placed or aligned on the bottles 130. The third output conveyer arrangement 111 removes any bottles 130 which have been incorrectly labeled as determined by the inspecting device.

The beverage bottling plant can be controlled by a central control arrangement 112, which could be, for example, computerized control system that monitors and controls the operation of the various stations and mechanisms of the beverage bottling plant.

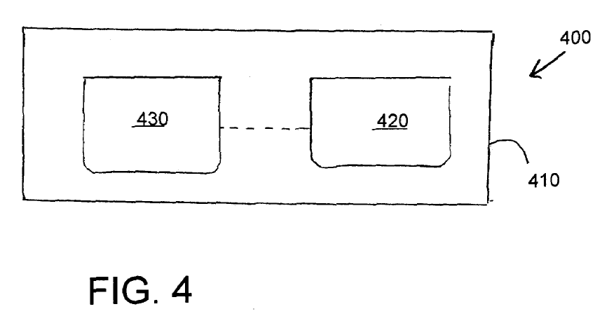

FIG. 4 schematically shows an embodiment of a treatment apparatus 400 for the treatment of bottles, cans, or similar containers. Electromagnetic energy source 420 may be any type of electromagnetic energy as is known in the art. For example, electromagnetic energy source 420 may be a microwave generator such as a magnetron, a Roentgen wave generator, or another type of electromagnetic energy generator. Electromagnetic energy source 420 may have a guide such as a wave guide configured to direct the electromagnetic energy in a desired direction, for example, toward cleaning machine 430.

Cleaning machine 430 is configured to expose bottles, cans, or other containers having an external source activatable treatment medium disposed therein to the electromagnetic energy emitted by electromagnetic energy source 420. In at least one possible embodiment, cleaning machine 430 has a rotatable rotor with a plurality of bottles, cans, or other containers proximate a periphery thereof that are passed through the electromagnetic energy emitted from electromagnetic energy source 420. In another possible embodiment, the cleaning machine 430 comprises a belt, holders, or other mechanisms configured to dispose the bottles, cans, or other containers in a substantially linear fashion to the electromagnetic energy emitted from electromagnetic energy source 420.

Electromagnetic energy source 420 may be disposed above, below, beside, or within cleaning machine 430. The configuration and orientation of electromagnetic energy source 420 with respect to cleaning machine 430 is such that the bottles, cans, or other containers having an external source activatable treatment medium disposed therein are exposed to the electromagnetic energy produced by electromagnetic energy source 420.

Electromagnetic energy shield 410 is shown in FIG. 4 disposed about electromagnetic energy source 420 and cleaning machine 430. Electromagnetic energy shield 410 may be comprised of any material configured to deflect, absorb, or otherwise prevent, restrict or minimize electromagnetic energy produced by electromagnetic energy source 420 from leaking into the ambient environment. The electromagnetic energy shield 410 is configured to substantially reduce leakage of electromagnetic energy produced by electromagnetic source 420 from leaking from treatment apparatus 400 while allowing bottles, cans, or other containers to be continuously or substantially continuously introduced into and exited from treatment apparatus 400.

The electromagnetic energy generated by electromagnetic source 420 may have any wave length and may be of any intensity to effectuate the activation of the activatable treatment medium disposed within the bottles, cans, or other containers. For example, microwaves which may have wavelengths ranging from one millimeter to one meter, or frequencies between 0.3 GHz and three hundred GHz may be generated. Alternatively or additionally, Roentgen waves or X-rays having a wavelength in the range of ten to 0.001 nanometers, which may correspond to frequencies in the range 30 petahertz to 30 exahertz (30×10¹⁵ Hz to 30×10¹⁸ Hz) and may have energies in the range one hundred twenty eV to one hundred twenty keV may be generated. Further, electromagnetic energy generated by electromagnetic source 420 may have wave lengths and frequencies throughout the electromagnetic energy spectrum, including but not limited to Roentgen and microwaves. At least a portion of the electromagnetic energy generated by electromagnetic source 420 has a wave length and frequency to activate the activatable treatment medium disposed within the bottles, cans, or other containers. In an embodiment wherein the electromagnetic source 420 is positioned outside of the bottles, cans, or other containers, the wave length and frequency of the electromagnetic energy is such that at least a portion of the electromagnetic energy passes through the bottle, can, or other container to activate the treatment medium within the bottle, can, or other container.

In other words and in conjunction with at least one possible embodiment according to the present application, the electromagnetic source 420 could generate electromagnetic energy falling either above or below the ranges listed in the above paragraph. For example, the electromagnetic source 420 could generate Roentgen waves or X-rays below thirty petahertz. The electromagnetic source 420 could also produce Roentgen waves or X-rays above 30 exahertz. Also, the electromagnetic source 420 may produce microwaves below 0.3 Ghz. In another possible embodiment, the electromagnetic source 420 may produce microwaves above 300 GHz.

Electromagnetic source 420 may comprise a wave guide configured to carry waves over a wide portion of the electromagnetic spectrum. The wave guide may be comprised of conductive materials, dielectric materials, or both.

Wave lengths and intensities of the electromagnetic energy generated by electromagnetic source 420 as well as the exposure time of the bottles, cans, or other containers having the external source activatable treatment medium disposed therein to the electromagnetic energy may be determined by experimentation. For example, a sweep generator may be used to produce electromagnetic energy and expose the energy to bottles, cans, or other containers having the external source activatable treatment medium disposed therein at varying frequencies, intensities, and durations. The amount of activation of the treatment medium may then be measured for the different frequencies, intensities, and durations to determine optimum conditions for different treatment mediums.

One feature or aspect of an embodiment is believed at the time of the filing of this patent application to possibly reside broadly in a method for the treatment, substantially for the sterilization, of bottles, cans or similar containers 2 by activation of a treatment medium in the respective container by the input of energy, wherein the activation is accomplished by activating the treatment medium with an electromagnetic radiation.

Another feature or aspect of an embodiment is believed at the time of the filing of this patent application to possibly reside broadly in the method, wherein the electromagnetic radiation is a radiation in the microwave range.

Yet another feature or aspect of an embodiment is believed at the time of the filing of this patent application to possibly reside broadly in the method, wherein the electromagnetic radiation is an X-ray radiation.

Still another feature or aspect of an embodiment is believed at the time of the filing of this patent application to possibly reside broadly in the method, comprising a mixture of at least two treatment media.

A further feature or aspect of an embodiment is believed at the time of the filing of this patent application to possibly reside broadly in the method, wherein the treatment mixture is a mixture of at least one gaseous and/or vapor component and a liquid component which is finely divided in the gaseous and/or vapor component.

Another feature or aspect of an embodiment is believed at the time of the filing of this patent application to possibly reside broadly in the method, wherein the at least one treatment medium comprises hydrogen peroxide (H₂O₂), often hydrogen peroxide in a solvent, for example in aqueous solution.

Yet another feature or aspect of an embodiment is believed at the time of the filing of this patent application to possibly reside broadly in the method, wherein the treatment medium contains hydrogen peroxide in aqueous solution finely distributed in a vapor and/or gaseous component, for example in air.

Still another feature or aspect of an embodiment is believed at the time of the filing of this patent application to possibly reside broadly in the method, wherein the treatment medium is water or steam.

A further feature or aspect of an embodiment is believed at the time of the filing of this patent application to possibly reside broadly in the method, wherein the frequency spectrum of the electromagnetic radiation is selected so that at least one frequency of this radiation is equal or approximately equal to the resonance frequency of at least one component of the treatment medium or a multiple or a fraction of this resonance frequency.

Another feature or aspect of an embodiment is believed at the time of the filing of this patent application to possibly reside broadly in the method, wherein at the at least one treatment medium is introduced into the individual container.

Yet another feature or aspect of an embodiment is believed at the time of the filing of this patent application to possibly reside broadly in the method, wherein the treatment medium is a medium that is diffused or incorporated into the container wall.

One feature or aspect of an embodiment is believed at the time of the filing of this patent application to possibly reside broadly in an apparatus for the treatment, such as for the sterilization, of bottles, cans or similar containers 2 by activation of a treatment medium in the respective container by the input of energy, with at least one transport element 5 that moves the container during the treatment on a transport line, wherein at least one generator 12 for the generation of an electromagnetic radiation directed at the individual container 2 is provided for the activation of the treatment medium in the respective container.

Another feature or aspect of an embodiment is believed at the time of the filing of this patent application to possibly reside broadly in the apparatus, comprising means 8, 10 for the introduction of at least one treatment medium into the container.

Yet another feature or aspect of an embodiment is believed at the time of the filing of this patent application to possibly reside broadly in the apparatus, wherein the means for the introduction of the at least one treatment medium are formed by a treatment head 8 which is provided in each treatment station.

Still another feature or aspect of an embodiment is believed at the time of the filing of this patent application to possibly reside broadly in the apparatus, wherein the at least one generator 12 is realized for the generation of an electromagnetic radiation in the microwave range.

A further feature or aspect of an embodiment is believed at the time of the filing of this patent application to possibly reside broadly in the apparatus, wherein the at least one generator 12 is realized for the generation of X-ray radiation.

Another feature or aspect of an embodiment is believed at the time of the filing of this patent application to possibly reside broadly in the apparatus, wherein the at least one generator 12 for the generation of the electromagnetic radiation is installed in a stationary position on the path of movement of the container 5 that is moved with the transport element 5.

Yet another feature or aspect of an embodiment is believed at the time of the filing of this patent application to possibly reside broadly in the apparatus, wherein the at least one generator 12 for the generation of the electromagnetic radiation is provided on the transport element 5 and is moved with the transport element.

Still another feature or aspect of an embodiment is believed at the time of the filing of this patent application to possibly reside broadly in the apparatus, wherein the transport line is formed by a plurality of transport elements that are arranged one after another in the direction of transport.

A further feature or aspect of an embodiment is believed at the time of the filing of this patent application to possibly reside broadly in the apparatus, wherein the at least one transport element is a rotor 5 that is driven in rotation around a vertical machine axis.

Another feature or aspect of an embodiment is believed at the time of the filing of this patent application to possibly reside broadly in a method of bottling juice in blow molded bottles comprising: conveying blow molded bottles to a first star wheel with a first conveyor; moving the blow molded bottles into a blow molded bottle cleaning machine from said first conveyor with said first star wheel; treating the blow molded bottles in said cleaning machine by a method comprising the further steps of: disposing an external source activatable treatment medium into an interior portion of the blow molded bottles; and activating, from a source external of the blow molded bottles, said external source activatable treatment medium while minimizing deformation of the blow molded bottles; said method of bottling juice further comprising: moving the blow molded bottles out of said blow molded bottle cleaning machine with a second star wheel; conveying the blow molded bottles to a third star wheel with a second conveyor; moving the blow molded bottles into a blow molded bottle filling machine with said third star wheel; filling the blow molded bottles with juice; moving the filled blow molded bottles out of said blow molded bottle filling machine with a fourth star wheel; conveying the filled blow molded bottles with a third conveyor to a fifth star wheel; moving the filled blow molded bottles into a blow molded bottle closing machine with said fifth star wheel; closing the filled blow molded bottles; and moving the closed blow molded bottles out of said blow molded bottle closing machine with a sixth star wheel.

Another feature or aspect of an embodiment is believed at the time of the filing of this patent application to possibly reside broadly in an apparatus configured to operate according to the method, said apparatus comprising: an arrangement configured to convey blow molded bottles to a first star wheel with a first conveyor; an arrangement configured to move the blow molded bottles into a blow molded bottle cleaning machine from said first conveyor with said first star wheel; an arrangement configured to treat the blow molded bottles comprising: an arrangement configured to dispose an external source activatable treatment medium into an interior portion of the blow molded bottles; and an arrangement configured to activate, from a source external of the blow molded bottles, said external source activatable treatment medium while minimizing deformation of the blow molded bottles; said apparatus further comprising: an arrangement configured to move the blow molded bottles out of said blow molded bottle cleaning machine with a second star wheel; an arrangement configured to convey the blow molded bottles to a third star wheel with a second conveyor; an arrangement configured to move the blow molded bottles into a blow molded bottle filling machine with said third star wheel; an arrangement configured to fill the blow molded bottles with juice; an arrangement configured to move the filled blow molded bottles out of said blow molded bottle filling machine with a fourth star wheel; an arrangement configured to convey the filled blow molded bottles with a third conveyor to a fifth star wheel; an arrangement configured to move the filled blow molded bottles into a blow molded bottle closing machine with said fifth star wheel; an arrangement configured to close the filled blow molded bottles; and an arrangement configured to move the closed blow molded bottles out of said blow molded bottle closing machine with a sixth star wheel.

Another feature or aspect of an embodiment is believed at the time of the filing of this patent application to possibly reside broadly in the method, wherein: said step of activating said external source activatable treatment medium in the blow molded bottles comprises activating said external source activatable treatment medium in the blow molded bottles with electromagnetic radiation; and said step of disposing an external source activatable treatment medium into an interior portion of the blow molded bottles comprises at least one of a), b), c) and d): a) disposing a treatment medium comprising a mixture of at least two treatment media; b) disposing a treatment medium comprising at least one vapor component; c) disposing a treatment medium comprising hydrogen peroxide; and d) disposing a treatment medium comprising water.

Another feature or aspect of an embodiment is believed at the time of the filing of this patent application to possibly reside broadly in the method, wherein: said step of activating said external source activatable treatment medium in the blow molded bottles comprises activating said external source activatable treatment medium in the blow molded bottles with electromagnetic radiation in the microwave or Roentgen range; said step of disposing an external source activatable treatment medium into an interior portion of the blow molded bottles comprises all of a), b), c) and d): a) disposing a treatment medium comprising a mixture of at least two treatment media; b) disposing a treatment medium comprising at least one vapor component; c) disposing a treatment medium comprising hydrogen peroxide; and d) disposing a treatment medium comprising water; and said step of treating the blow molded bottles being performed by a blow molded bottle cleaning machine, said blow molded bottle cleaning machine comprising: a rotor; a rotatable vertical machine column; said rotor being connected to said vertical machine column to permit rotation of said rotor about said vertical machine column; a plurality of holding elements disposed proximate a periphery of said rotor configured to hold said bottles, cans or similar containers; at least one dispensing arrangement being configured and disposed to dispense a treatment material into an interior portion of said bottles, cans or similar containers through an opening therein; an activator disposed sufficiently close to said holding elements to activate said treatment material; and a controller configured to control said activator.

Another feature or aspect of an embodiment is believed at the time of the filing of this patent application to possibly reside broadly in a method of filling bottles, cans or similar containers comprising: conveying bottles, cans or similar containers into a container cleaning machine; cleaning an interior portion of the bottles, cans or similar containers, said cleaning comprising the steps of: disposing an electromagnetically activatable treatment medium into an interior portion of the bottles, cans or similar containers; and activating said electromagnetically activatable treatment medium in the bottles, cans or similar containers with an electromagnetic activation source while minimizing deformation of the bottles, cans or similar containers; said method of filling further comprising the steps of: conveying the bottles, cans or similar containers out of said container cleaning machine and into a container filling machine; filling the bottles, cans or similar containers; conveying the bottles, cans or similar containers out of said container filling machine and into a container closing machine; closing the filled bottles, cans or similar containers; and conveying the closed bottles, cans or similar containers out of said container closing machine.

Another feature or aspect of an embodiment is believed at the time of the filing of this patent application to possibly reside broadly in the method, wherein: said step of activating said electromagnetically activatable treatment medium in the bottles, cans or similar containers comprises activating said electromagnetically activatable treatment medium in the bottles, cans or similar containers with electromagnetic radiation in the microwave range; and said step of disposing an electromagnetically activatable treatment medium into an interior portion of the bottles, cans or similar containers comprises at least one of a), b), c) and d): a) disposing a treatment medium comprising a mixture of at least two treatment media into an interior portion of the bottles, cans or similar containers; b) disposing a treatment medium comprising at least one vapor component into an interior portion of the bottles, cans or similar containers; c) disposing a treatment medium comprising hydrogen peroxide into an interior portion of the bottles, cans or similar containers; and d) disposing a treatment medium comprising water into an interior portion of the bottles, cans or similar containers.

Another feature or aspect of an embodiment is believed at the time of the filing of this patent application to possibly reside broadly in the method, wherein: said step of activating said electromagnetically activatable treatment medium in the bottles, cans or similar containers comprises activating said electromagnetically activatable treatment medium in the bottles, cans or similar containers with electromagnetic radiation in the microwave range; and said step of disposing an electromagnetically activatable treatment medium into an interior portion of the bottles, cans or similar containers comprises all of a), b), c) and d): a) disposing a treatment medium comprising a mixture of at least two treatment media into an interior portion of the bottles, cans or similar containers; b) disposing a treatment medium comprising at least one vapor component into an interior portion of the bottles, cans or similar containers; c) disposing a treatment medium comprising hydrogen peroxide into an interior portion of the bottles, cans or similar containers; and d) disposing a treatment medium comprising water into an interior portion of the bottles, cans or similar containers.

Another feature or aspect of an embodiment is believed at the time of the filing of this patent application to possibly reside broadly in the method, wherein: said step of cleaning an interior portion of the bottles, cans or similar containers is performed by a container cleaning machine, said container machine comprising: a rotor; a rotatable vertical machine column; said rotor being connected to said vertical machine column to permit rotation of said rotor about said vertical machine column; and a plurality of holding elements disposed proximate a periphery of said rotor configured to hold said bottles, cans or similar containers; said step of disposing an electromagnetically activatable treatment medium into an interior portion of the bottles, cans or similar containers is performed by a dispensing apparatus, said dispensing apparatus comprising at least one dispensing arrangement being configured and disposed to dispense the electromagnetically activatable treatment medium into the interior portion of the bottles, cans or similar containers; said step of activating said electromagnetically activatable treatment medium in the bottles, cans or similar containers with an electromagnetic activation source while minimizing deformation of the bottles, cans or similar containers is performed by an an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency; said activator is disposed sufficiently close to said holding elements to activate said treatment material; and a controller configured to control said activator.

Another feature or aspect of an embodiment is believed at the time of the filing of this patent application to possibly reside broadly in the method, wherein: said step of activating said electromagnetically activatable treatment medium in the bottles, cans or similar containers comprises activating said electromagnetically activatable treatment medium in the bottles, cans or similar containers with electromagnetic radiation in the Roentgen; and said step of disposing an electromagnetically activatable treatment medium into an interior portion of the bottles, cans or similar containers comprises at least one of a), b), c) and d): a) disposing a treatment medium comprising a mixture of at least two treatment media into an interior portion of the bottles, cans or similar containers; b) disposing a treatment medium comprising at least one vapor component into an interior portion of the bottles, cans or similar containers; c) disposing a treatment medium comprising hydrogen peroxide into an interior portion of the bottles, cans or similar containers; and d) disposing a treatment medium comprising water into an interior portion of the bottles, cans or similar containers.

Another feature or aspect of an embodiment is believed at the time of the filing of this patent application to possibly reside broadly in the method, wherein: said step of activating said electromagnetically activatable treatment medium in the bottles, cans or similar containers comprises activating said electromagnetically activatable treatment medium in the bottles, cans or similar containers with electromagnetic radiation in the Roentgen range; and said step of disposing an electromagnetically activatable treatment medium into an interior portion of the bottles, cans or similar containers comprises all of a), b), c) and d): a) disposing a treatment medium comprising a mixture of at least two treatment media into an interior portion of the bottles, cans or similar containers; b) disposing a treatment medium comprising at least one vapor component into an interior portion of the bottles, cans or similar containers; c) disposing a treatment medium comprising hydrogen peroxide into an interior portion of the bottles, cans or similar containers; and d) disposing a treatment medium comprising water into an interior portion of the bottles, cans or similar containers.

Another feature or aspect of an embodiment is believed at the time of the filing of this patent application to possibly reside broadly in the method, wherein: said step of cleaning an interior portion of the bottles, cans or similar containers is performed by a container cleaning machine, said container machine comprising: a rotor; a rotatable vertical machine column; said rotor being connected to said vertical machine column to permit rotation of said rotor about said vertical machine column; and a plurality of holding elements disposed proximate a periphery of said rotor configured to hold said bottles, cans or similar containers; said step of disposing an electromagnetically activatable treatment medium into an interior portion of the bottles, cans or similar containers is performed by a dispensing apparatus, said dispensing apparatus comprising at least one dispensing arrangement being configured and disposed to dispense the electromagnetically activatable treatment medium into the interior portion of the bottles, cans or similar containers; said step of activating said electromagnetically activatable treatment medium in the bottles, cans or similar containers with an electromagnetic activation source while minimizing deformation of the bottles, cans or similar containers is performed by an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency; said activator disposed sufficiently close to said holding elements to activate said treatment material; and a controller configured to control said activator.

Another feature or aspect of an embodiment is believed at the time of the filing of this patent application to possibly reside broadly in an apparatus configured to operate according to the method, said apparatus comprising: an arrangement configured to convey bottles, cans or similar containers into a container cleaning machine; an arrangement configured to clean an interior portion of the bottles, cans or similar containers, said arrangement configured to clean comprising: an arrangement configured to dispose an electromagnetically activatable treatment medium into an interior portion of the bottles, cans or similar containers; and an arrangement configured to activate said electromagnetically activatable treatment medium in the bottles, cans or similar containers with an electromagnetic activation source while minimizing deformation of the bottles, cans or similar containers; said apparatus further comprising: an arrangement configured to convey the bottles, cans or similar containers out of said container cleaning machine and into a container filling machine; an arrangement configured to fill the bottles, cans or similar containers; an arrangement configured to convey the bottles, cans or similar containers out of said container filling machine and into a container closing machine; an arrangement configured to close the filled bottles, cans or similar containers; and an arrangement configured to convey the closed bottles, cans or similar containers out of said container closing machine.

Another feature or aspect of an embodiment is believed at the time of the filing of this patent application to possibly reside broadly in an apparatus configured to operate according to the method, said apparatus comprising: an arrangement configured to convey bottles, cans or similar containers into a container cleaning machine; an arrangement configured to clean an interior portion of the bottles, said arrangement configured to clean comprising: a rotatable rotor; a plurality of holding elements disposed proximate a periphery of said rotor configured to hold said bottles, cans or similar containers; an arrangement configured to dispose an electromagnetically activatable treatment medium into an interior portion of the bottles, cans or similar containers; and an arrangement configured to activate said electromagnetically activatable treatment medium in the bottles, cans or similar containers with an electromagnetic activation source while minimizing deformation of the bottles, cans or similar containers comprising: a magnetron configured to generate electromagnetic radiation; a wave guide disposed about a portion of said rotor configured to activate said electromagnetically activatable treatment medium in the bottles, cans or similar containers with said electromagnetic radiation; a controller configured to control said magnetron; said apparatus further comprising: an arrangement configured to convey the bottles, cans or similar containers out of said container cleaning machine and into a container filling machine; an arrangement configured to fill the bottles, cans or similar containers; an arrangement configured to convey the bottles, cans or similar containers out of said container filling machine and into a container closing machine; an arrangement configured to close the filled bottles, cans or similar containers; and an arrangement configured to convey the closed bottles, cans or similar containers out of said container closing machine; said arrangement configured to activate said electromagnetically activatable treatment medium in the bottles, cans or similar containers comprising an arrangement configured to activate said electromagnetically activatable treatment medium in the bottles, cans or similar containers with electromagnetic radiation in the microwave range; and said arrangement configured to dispose said electromagnetically activatable treatment medium into an interior portion of the bottles, cans or similar containers comprises all of a), b), c) and d): a) an arrangement configured to dispose a treatment medium comprising a mixture of at least two treatment media into an interior portion of the bottles, cans or similar containers; b) an arrangement configured to dispose a treatment medium comprising at least one vapor component into an interior portion of the bottles, cans or similar containers; c) an arrangement configured to dispose a treatment medium comprising hydrogen peroxide into an interior portion of the bottles, cans or similar containers; and d) an arrangement configured to dispose a treatment medium comprising water into an interior portion of the bottles, cans or similar containers.

Another feature or aspect of an embodiment is believed at the time of the filing of this patent application to possibly reside broadly in an apparatus configured to operate according to the method, said apparatus comprising: an arrangement configured to convey bottles, cans or similar containers into a container cleaning machine; an arrangement configured to clean an interior portion of the bottles, said arrangement configured to clean comprising: a rotatable rotor; a plurality of holding elements disposed proximate a periphery of said rotor configured to hold said bottles, cans or similar containers; an arrangement configured to dispose an electromagnetically activatable treatment medium into an interior portion of the bottles, cans or similar containers; and an arrangement configured to activate said electromagnetically activatable treatment medium in the bottles, cans or similar containers with an electromagnetic activation source while minimizing deformation of the bottles, cans or similar containers comprising: a Roentgen generator configured to generate electromagnetic radiation; a Roentgen wave guide disposed about a portion of said rotor configured to activate said electromagnetically activatable treatment medium in the bottles, cans or similar containers with said electromagnetic radiation; and a controller configured to control said magnetron; an arrangement configured to convey the bottles, cans or similar containers out of said container cleaning machine and into a container filling machine; an arrangement configured to fill the bottles, cans or similar containers; an arrangement configured to convey the bottles, cans or similar containers out of said container filling machine and into a container closing machine; an arrangement configured to close the filled bottles, cans or similar containers; and an arrangement configured to convey the closed bottles, cans or similar containers out of said container closing machine; said arrangement configured to activate said electromagnetically activatable treatment medium in the bottles, cans or similar containers comprising an arrangement configured to activate said electromagnetically activatable treatment medium in the bottles, cans or similar containers with electromagnetic radiation in the Roentgen range; and said arrangement configured to dispose said electromagnetically activatable treatment medium into an interior portion of the bottles, cans or similar containers comprising all of a), b), c) and d): a) an arrangement configured to dispose a treatment medium comprising a mixture of at least two treatment media into an interior portion of the bottles, cans or similar containers; b) an arrangement configured to dispose a treatment medium comprising at least one vapor component into an interior portion of the bottles, cans or similar containers; c) an arrangement configured to dispose a treatment medium comprising hydrogen peroxide into an interior portion of the bottles, cans or similar containers; and d) an arrangement configured to dispose a treatment medium comprising water into an interior portion of the bottles, cans or similar containers.

Another feature or aspect of an embodiment is believed at the time of the filing of this patent application to possibly reside broadly in the method, wherein: said step of activating said electromagnetically activatable treatment medium in the bottles, cans or similar containers with an electromagnetic activation source while minimizing deformation of the bottles, cans or similar containers is performed by an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency.

Another feature or aspect of an embodiment is believed at the time of the filing of this patent application to possibly reside broadly in the method, wherein: said step of activating said electromagnetically activatable treatment medium in the bottles, cans or similar containers with an electromagnetic activation source while minimizing deformation of the bottles, cans or similar containers is performed by an an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency.

Another feature or aspect of an embodiment is believed at the time of the filing of this patent application to possibly reside broadly in the method, wherein: said container cleaning machine comprises: a rotatable rotor; a plurality of holding elements disposed proximate a periphery of said rotor configured to hold said bottles, cans or similar containers; at least one dispensing arrangement being configured and disposed to dispense a treatment material into an interior portion of said bottles, cans or similar containers through an opening therein; a magnetron configured to generate electromagnetic radiation; a wave guide disposed about a portion of said rotor configured to activate said said electromagnetically activatable treatment medium in the bottles, cans or similar containers with said electromagnetic radiation; and a controller configured to control said magnetron.

Another feature or aspect of an embodiment is believed at the time of the filing of this patent application to possibly reside broadly in a means for filling bottles, cans or similar containers comprising: means for cleaning an interior portion of bottles, cans or similar containers; said means for cleaning an interior portion of bottles, cans or similar containers comprising: means for disposing an electromagnetically activatable treatment medium into an interior portion of the bottles, cans or similar containers; means for activating said electromagnetically activatable treatment medium in the bottles, cans, or similar containers with an electromagnetic activation source while minimizing deformation of the bottles, cans or similar containers; means for conveying bottles, cans or similar containers into said means for cleaning an interior portion of the bottles, cans or similar containers; means for filling bottles, cans or similar containers; means for conveying bottles, cans or similar containers out of said means for cleaning an interior portion of bottles, cans or similar containers and into said means for filling bottles, cans or similar containers; means for closing bottles, cans or similar containers; means for conveying filled bottles, cans or similar containers out of said means for filling bottles, cans or similar containers and into said means for closing bottles, cans or similar containers; means for conveying closed bottles, cans or similar containers out of said means for closing bottles, cans or similar containers.

Another feature or aspect of an embodiment is believed at the time of the filing of this patent application to possibly reside broadly in the means, wherein: said means for activating said electromagnetically activatable treatment medium in the bottles, cans, or similar containers with an electromagnetic activation source while minimizing deformation of the bottles, cans or similar containers comprises means for activating said electromagnetically activatable treatment medium in the bottles, cans or similar containers with electromagnetic radiation in the microwave range; and said means for disposing an electromagnetically activatable treatment medium into an interior portion of the bottles, cans or similar containers comprises all of a), b), c) and d): a) means for disposing a treatment medium comprising a mixture of at least two treatment media into an interior portion of the bottles, cans or similar containers; b) means for disposing a treatment medium comprising at least one vapor component into an interior portion of the bottles, cans or similar containers; c) means for disposing a treatment medium comprising hydrogen peroxide into an interior portion of the bottles, cans or similar containers; and d) means for disposing a treatment medium comprising water into an interior portion of the bottles, cans or similar containers.

Another feature or aspect of an embodiment is believed at the time of the filing of this patent application to possibly reside broadly in an arrangement configured to fill bottles, cans, or similar containers, wherein: said means for activating said electromagnetically activatable treatment medium in the bottles, cans, or similar containers with an electromagnetic activation source while minimizing deformation of the bottles, cans or similar containers comprises means for activating said electromagnetically activatable treatment medium in the bottles, cans or similar containers with electromagnetic radiation in the Roentgen range; and said means for disposing an electromagnetically activatable treatment medium into an interior portion of the bottles, cans or similar containers comprises all of a), b), c) and d): a) means for disposing a treatment medium comprising a mixture of at least two treatment media into an interior portion of the bottles, cans or similar containers; b) means for disposing a treatment medium comprising at least one vapor component into an interior portion of the bottles, cans or similar containers; c) means for disposing a treatment medium comprising hydrogen peroxide into an interior portion of the bottles, cans or similar containers; and d) means for disposing a treatment medium comprising water into an interior portion of the bottles, cans or similar containers.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that any or all the examples of patents, published patent applications, and other documents which are included in this application and including those which are referred to in paragraphs which state “Some examples of . . . which may possibly be used in at least one possible embodiment of the present application . . . ” may possibly not be used or useable in any one or more or any embodiments of the application.

The sentence immediately above relates to patents, published patent applications and other documents either incorporated by reference or not incorporated by reference.

Some examples of decomposition and other features which may possibly be utilizable by at least one possible embodiment may possibly be found in the following: “THE RADIATION-INDUCED DECOMPOSITION OF MILLIMOLAR CONCENTRATIONS OF HYDROGEN PEROXIDE IN AERATED ‘PURE WATER”, by Balkwell, William R. and Oldham, Susan B. 19 Aug. 1965; “Dissociation of water by microwave radiation”, by V. L. Vaks, G. A. Domrachev, Yu. L. Rodygin, D. A. Selivanovskii and E. I. Spivak, 28 Jul. 1993; “PLASMA CHEMISTRY AND PLASMA PROCESSING”, Vols. 1-26. New York, 1981-2006; “H₂O₂ Thermodynamic Properties” at http://www.h2o2.com/intro/properties/thermodynamic.html, visited Dec. 1, 2008; “Combined effects of microwave heating and hydrogen peroxide on the destruction of Escherichia coli”, KOUTCHMA T. and RAMASWAMY H. S., 2000; “Features of sterilization using low-pressure DC-discharge hydrogen-peroxide plasma”, by Soloshenko, I. A.; Tsiolko, V. V.; Khomich, V. A.; Bazhenov, V. Yu.; Ryabtsev, A. V.; Schedrin, A. I.; Mikhno, I. L., August 2002; U.S. Pat. No. 3,739,225, having inventor James R. MIMS; U.S. Pat. No. 4,020,311, having inventor Mark T. CHURCHLAND; U.S. Pat. No. 3,946,236, having inventors Thomas G. ROBERTS, et al.; U.S. Pat. No. 5,651,045, having inventors Jean-Michel POUVESLE, et al.; U.S. Pat. No. 6,529,579, having inventor John RICHARDSON; U.S. Pat. No. 5,015,971, having inventors Stephen D. TAYLOR, et al.; and U.S. Pat. No. 5,961,871, having inventors Don W. BIBLE, et al.

Some examples of a electromagnetic wave guides that may possibly be utilized or adapted for use in at least one possible embodiment may possibly be found in the following U.S. patents: U.S. Pat. No. 7,368,692, having inventor Abdulkadir HALLAC; and U.S. Pat. No. 5,103,103, having inventors Kenneth C. RADFORD, et al.

Some examples of a electromagnetic shields that may possibly be utilized or adapted for use in at least one possible embodiment may possibly be found in the following U.S. Pat. No. 5,103,103, having inventors Kenneth C. RADFORD, et al; U.S. Pat. No. 6,718,008, having inventors Bob Baoping H E, et al.; U.S. Pat. No. 5,981,964, having inventors Bruce J. MCAULEY, et al.; and U.S. Pat. No. 4,158,779, having inventors Werner ROMMEL, et al.

The following patents, patent applications or patent publications, are hereby incorporated by reference as if set forth in their entirety herein: DE 10 2004 030 957, having the English translation of the German title “METHOD OF STERILISING BOTTLES OR SIMILAR CONTAINERS, AND STERILISER FOR CARRYING OUT THE METHOD,” published on Jan. 12, 2006; and DE 10 2004 030 956, having the English translation of the German title “STERILIZATION OF CONTAINERS, ESPECIALLY PLASTIC BOTTLES FOR DRINKS BY BLOWING HYDROGEN PEROXIDE VAPOR AND HOT AIR THEN BLOWING AIR AT SUFFICIENTLY HIGH FLOW RATE TO AVOID EXCESSIVE CONTAINER TEMPERATURE,” published on Jan. 12, 2006.

All of the patents, patent applications or patent publications, which were cited in the International Search Report dated Aug. 30, 2008, and/or cited elsewhere are hereby incorporated by reference as if set forth in their entirety herein as follows: DE 40 18 142, having the following English translation of the German title “METHOD FOR FILLING BOTTLES WITH BEVERAGES—INVOLVES MICROWAVE RADIATION OF BOTTLES PRIOR TO FILLING”, published on Dec. 12, 1991; U.S. Pat. No. 3,780,308, having the title “PROCESS AND APPARATUS FOR SURFACE STERILIZATION OF MATERIALS”, published on Dec. 18, 1973; WO 99/21593, having the title “METHOD AND APPARATUS FOR STERILIZING A PACKAGING SHEET MATERIAL”, published on May 6, 1999; DE 10 2004 030956, having the following English translation of the German title “STERILIZATION OF CONTAINERS, ESPECIALLY PLASTICS BOTTLES FOR DRINKS, BY BLOWING IN HYDROGEN PEROXIDE VAPOR AND HOT AIR THEN BLOWING IN AIR AT SUFFICIENTLY HIGH FLOW RATE TO AVOID EXCESSIVE CONTAINER TEMPERATURE”, published on Jan. 12, 2006; DE 10 54 910, having the German title “MASCHINENGRUPPE ZUM FORTLAUFENDEN BEHANDELN VON GEFAESSEN”, published on May 13, 1992; EP 0 484 730, having the title “METHOD OF STERILIZING A PACKAGING MATERIAL BY MEANS OF A STERILIZING AGENT IN LIQUID FORM”, published on May 13, 1992; and US2005/260096, having the title “METHOD AND APPARATUS FOR VAPORIZING A STERILANT FLUID USING MICROWAVE ENERGY”, published on Nov. 24, 2005.

The patents, patent applications, and patent publications listed above, beginning on line 1297 on page 49 in the paragraph with the statement: “Some examples of decomposition and other features which may be utilizable by at least one possible embodiment . . . ” and ending on line 1372 on page 51 in the paragraph with the phrase: “ . . . and US2005/260096, having the title “METHOD AND APPARATUS FOR VAPORIZING A STERILANT FLUID USING MICROWAVE ENERGY”, published on Nov. 24, 2005,” are herein incorporated by reference as if set forth in their entirety. The purpose of incorporating U.S. patents, Foreign patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. 10 2006 026 278.6, filed on Jun. 2, 2006, having inventor Volker TILL, and DE-OS 10 2006 026 278.6 and DE-PS 10 2006 026 278.6, and International Application No. PCT/EP2007/004599, filed on May 24, 2007, having WIPO Publication No. WO 2007/140883 and inventor Volker TILL, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the Foreign equivalent patent application PCT/EP2007/004599 and German Patent Application 10 2006 026 278.6 is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2007/004599 and DE 10 2006 026 278.6 from which this patent application claims priority which do not have to do with the correction of the translation in this patent application are not to be included in this patent application in the incorporation by reference.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

### AT LEAST PARTIAL NOMENCLATURE

1 Apparatus for sterilization
2 Bottles
2.1 Bottle mouth
2.2 Flange
3 Conveyor
4 Transport star wheel
5 Rotor
6 Treatment station
7 Bottle or container carrier
8 Treatment head
9 Outlet star wheel
10 Treatment tube
11 Activator
12 Generator for the generation of the electromagnetic alternating field

What is claimed is:

1. A method of bottling juice in blow molded bottles comprising:
conveying blow molded bottles to a first star wheel with a first conveyor;
moving the blow molded bottles into a blow molded bottle cleaning machine from said first conveyor with said first star wheel;
treating the blow molded bottles in said cleaning machine by a method comprising the further steps of:
disposing an external source activatable treatment medium into an interior portion of the blow molded bottles; and
activating, from a source external of the blow molded bottles, said external source activatable treatment medium while minimizing deformation of the blow molded bottles;
said method of bottling juice further comprising:
moving the blow molded bottles out of said blow molded bottle cleaning machine with a second star wheel;
conveying the blow molded bottles to a third star wheel with a second conveyor;
moving the blow molded bottles into a blow molded bottle filling machine with said third star wheel;
filling the blow molded bottles with juice;
moving the filled blow molded bottles out of said blow molded bottle filling machine with a fourth star wheel;
conveying the filled blow molded bottles with a third conveyor to a fifth star wheel;
moving the filled blow molded bottles into a blow molded bottle closing machine with said fifth star wheel;
closing the filled blow molded bottles; and
moving the closed blow molded bottles out of said blow molded bottle closing machine with a sixth star wheel.

2. An apparatus configured to operate according to the method of claim 1, said apparatus comprising:
an arrangement configured to convey blow molded bottles to a first star wheel with a first conveyor;
an arrangement configured to move the blow molded bottles into a blow molded bottle cleaning machine from said first conveyor with said first star wheel;
an arrangement configured to treat the blow molded bottles comprising:
an arrangement configured to dispose an external source activatable treatment medium into an interior portion of the blow molded bottles; and
an arrangement configured to activate, from a source external of the blow molded bottles, said external source activatable treatment medium while minimizing deformation of the blow molded bottles;
said apparatus further comprising:
an arrangement configured to move the blow molded bottles out of said blow molded bottle cleaning machine with a second star wheel;
an arrangement configured to convey the blow molded bottles to a third star wheel with a second conveyor;
an arrangement configured to move the blow molded bottles into a blow molded bottle filling machine with said third star wheel;
an arrangement configured to fill the blow molded bottles with juice;
an arrangement configured to move the filled blow molded bottles out of said blow molded bottle filling machine with a fourth star wheel;
an arrangement configured to convey the filled blow molded bottles with a third conveyor to a fifth star wheel;
an arrangement configured to move the filled blow molded bottles into a blow molded bottle closing machine with said fifth star wheel;
an arrangement configured to close the filled blow molded bottles; and
an arrangement configured to move the closed blow molded bottles out of said blow molded bottle closing machine with a sixth star wheel.

3. The method of bottling juice in blow molded bottles of claim 1, wherein:
said step of activating said external source activatable treatment medium in the blow molded bottles comprises activating said external source activatable treatment medium in the blow molded bottles with electromagnetic radiation; and
said step of disposing an external source activatable treatment medium into an interior portion of the blow molded bottles comprises at least one of a), b), c) and d):
a) disposing a treatment medium comprising a mixture of at least two treatment media;
b) disposing a treatment medium comprising at least one vapor component;
c) disposing a treatment medium comprising hydrogen peroxide; and
d) disposing a treatment medium comprising water.

4. The method of bottling juice in blow molded bottles of claim 3, wherein:
said step of activating said external source activatable treatment medium in the blow molded bottles comprises activating said external source activatable treatment medium in the blow molded bottles with electromagnetic radiation in the microwave or Roentgen range;
said step of disposing an external source activatable treatment medium into an interior portion of the blow molded bottles comprises all of a), b), c) and d):
a) disposing a treatment medium comprising a mixture of at least two treatment media;
b) disposing a treatment medium comprising at least one vapor component;
c) disposing a treatment medium comprising hydrogen peroxide; and
d) disposing a treatment medium comprising water; and
said step of treating the blow molded bottles being performed by a blow molded bottle cleaning machine, said blow molded bottle cleaning machine comprising:
a rotor;
a rotatable vertical machine column;
said rotor being connected to said vertical machine column to permit rotation of said rotor about said vertical machine column;
a plurality of holding elements disposed proximate a periphery of said rotor configured to hold said bottles, cans or similar containers;
at least one dispensing arrangement being configured and disposed to dispense a treatment material into an interior portion of said bottles, cans or similar containers through an opening therein;
an activator disposed sufficiently close to said holding elements to activate said treatment material; and
a controller configured to control said activator.

5. A method of filling bottles, cans or similar containers comprising:
conveying bottles, cans or similar containers into a container cleaning machine;
cleaning an interior portion of the bottles, cans or similar containers, said cleaning comprising the steps of:
disposing an electromagnetically activatable treatment medium into an interior portion of the bottles, cans or similar containers; and
activating said electromagnetically activatable treatment medium in the bottles, cans or similar containers with an electromagnetic activation source while minimizing deformation of the bottles, cans or similar containers;
said method of filling further comprising the steps of:
conveying the bottles, cans or similar containers out of said container cleaning machine and into a container filling machine;
filling the bottles, cans or similar containers;
conveying the bottles, cans or similar containers out of said container filling machine and into a container closing machine;
closing the filled bottles, cans or similar containers; and
conveying the closed bottles, cans or similar containers out of said container closing machine.

6. The method for filling bottles, cans or similar containers of claim 5, wherein:
said step of activating said electromagnetically activatable treatment medium in the bottles, cans or similar containers comprises activating said electromagnetically activatable treatment medium in the bottles, cans or similar containers with electromagnetic radiation in the microwave range; and
said step of disposing an electromagnetically activatable treatment medium into an interior portion of the bottles, cans or similar containers comprises at least one of a), b), c) and d):
a) disposing a treatment medium comprising a mixture of at least two treatment media into an interior portion of the bottles, cans or similar containers;
b) disposing a treatment medium comprising at least one vapor component into an interior portion of the bottles, cans or similar containers;
c) disposing a treatment medium comprising hydrogen peroxide into an interior portion of the bottles, cans or similar containers; and
d) disposing a treatment medium comprising water into an interior portion of the bottles, cans or similar containers.

7. The method for filling bottles, cans or similar containers of claim 5, wherein:
said step of activating said electromagnetically activatable treatment medium in the bottles, cans or similar containers comprises activating said electromagnetically activatable treatment medium in the bottles, cans or similar containers with electromagnetic radiation in the microwave range; and
said step of disposing an electromagnetically activatable treatment medium into an interior portion of the bottles, cans or similar containers comprises all of a), b), c) and d):
a) disposing a treatment medium comprising a mixture of at least two treatment media into an interior portion of the bottles, cans or similar containers;
b) disposing a treatment medium comprising at least one vapor component into an interior portion of the bottles, cans or similar containers;
c) disposing a treatment medium comprising hydrogen peroxide into an interior portion of the bottles, cans or similar containers; and
d) disposing a treatment medium comprising water into an interior portion of the bottles, cans or similar containers.

8. The method for filling bottles, cans or similar containers of claim 7, wherein:
said step of cleaning an interior portion of the bottles, cans or similar containers is performed by a container cleaning machine, said container machine comprising:
a rotor;
a rotatable vertical machine column;
said rotor being connected to said vertical machine column to permit rotation of said rotor about said vertical machine column; and
a plurality of holding elements disposed proximate a periphery of said rotor configured to hold said bottles, cans or similar containers;
said step of disposing an electromagnetically activatable treatment medium into an interior portion of the bottles, cans or similar containers is performed by a dispensing apparatus, said dispensing apparatus comprising at least one dispensing arrangement being configured and disposed to dispense the electromagnetically activatable treatment medium into the interior portion of the bottles, cans or similar containers;
said step of activating said electromagnetically activatable treatment medium in the bottles, cans or similar containers with an electromagnetic activation source while minimizing deformation of the bottles, cans or similar containers is performed by an an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency;
said activator is disposed sufficiently close to said holding elements to activate said treatment material; and
a controller configured to control said activator.

9. The method for filling bottles, cans or similar containers of claim 5, wherein:
said step of activating said electromagnetically activatable treatment medium in the bottles, cans or similar containers comprises activating said electromagnetically activatable treatment medium in the bottles, cans or similar containers with electromagnetic radiation in the Roentgen; and
said step of disposing an electromagnetically activatable treatment medium into an interior portion of the bottles, cans or similar containers comprises at least one of a), b), c) and d):
a) disposing a treatment medium comprising a mixture of at least two treatment media into an interior portion of the bottles, cans or similar containers;
b) disposing a treatment medium comprising at least one vapor component into an interior portion of the bottles, cans or similar containers;
c) disposing a treatment medium comprising hydrogen peroxide into an interior portion of the bottles, cans or similar containers; and
d) disposing a treatment medium comprising water into an interior portion of the bottles, cans or similar containers.

10. The method for filling bottles, cans or similar containers of claim 5, wherein:
said step of activating said electromagnetically activatable treatment medium in the bottles, cans or similar containers comprises activating said electromagnetically activatable treatment medium in the bottles, cans or similar containers with electromagnetic radiation in the Roentgen range; and
said step of disposing an electromagnetically activatable treatment medium into an interior portion of the bottles, cans or similar containers comprises all of a), b), c) and d):
a) disposing a treatment medium comprising a mixture of at least two treatment media into an interior portion of the bottles, cans or similar containers;
b) disposing a treatment medium comprising at least one vapor component into an interior portion of the bottles, cans or similar containers;
c) disposing a treatment medium comprising hydrogen peroxide into an interior portion of the bottles, cans or similar containers; and
d) disposing a treatment medium comprising water into an interior portion of the bottles, cans or similar containers.

11. The method for filling bottles, cans or similar containers of claim 10, wherein:
said step of cleaning an interior portion of the bottles, cans or similar containers is performed by a container cleaning machine, said container machine comprising:
a rotor;
a rotatable vertical machine column;
said rotor being connected to said vertical machine column to permit rotation of said rotor about said vertical machine column; and
a plurality of holding elements disposed proximate a periphery of said rotor configured to hold said bottles, cans or similar containers;
said step of disposing an electromagnetically activatable treatment medium into an interior portion of the bottles, cans or similar containers is performed by a dispensing apparatus, said dispensing apparatus comprising at least one dispensing arrangement being configured and disposed to dispense the electromagnetically activatable treatment medium into the interior portion of the bottles, cans or similar containers;
said step of activating said electromagnetically activatable treatment medium in the bottles, cans or similar containers with an electromagnetic activation source while minimizing deformation of the bottles, cans or similar containers is performed by an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency;
said activator disposed sufficiently close to said holding elements to activate said treatment material; and
a controller configured to control said activator.

12. An apparatus configured to operate according to the method of claim 5, said apparatus comprising:
an arrangement configured to convey bottles, cans or similar containers into a container cleaning machine;
an arrangement configured to clean an interior portion of the bottles, cans or similar containers, said arrangement configured to clean comprising:
an arrangement configured to dispose an electromagnetically activatable treatment medium into an interior portion of the bottles, cans or similar containers; and
an arrangement configured to activate said electromagnetically activatable treatment medium in the bottles, cans or similar containers with an electromagnetic activation source while minimizing deformation of the bottles, cans or similar containers;
said apparatus further comprising:
an arrangement configured to convey the bottles, cans or similar containers out of said container cleaning machine and into a container filling machine;
an arrangement configured to fill the bottles, cans or similar containers;
an arrangement configured to convey the bottles, cans or similar containers out of said container filling machine and into a container closing machine;
an arrangement configured to close the filled bottles, cans or similar containers; and
an arrangement configured to convey the closed bottles, cans or similar containers out of said container closing machine.

13. An apparatus configured to operate according to the method of claim 5, said apparatus comprising:
an arrangement configured to convey bottles, cans or similar containers into a container cleaning machine;
an arrangement configured to clean an interior portion of the bottles, said arrangement configured to clean comprising:
a rotatable rotor;
a plurality of holding elements disposed proximate a periphery of said rotor configured to hold said bottles, cans or similar containers;
an arrangement configured to dispose an electromagnetically activatable treatment medium into an interior portion of the bottles, cans or similar containers; and
an arrangement configured to activate said electromagnetically activatable treatment medium in the bottles, cans or similar containers with an electromagnetic activation source while minimizing deformation of the bottles, cans or similar containers comprising:
a magnetron configured to generate electromagnetic radiation;
a wave guide disposed about a portion of said rotor configured to activate said said electromagnetically activatable treatment medium in the bottles, cans or similar containers with said electromagnetic radiation; and
a controller configured to control said magnetron;
said apparatus further comprising:
an arrangement configured to convey the bottles, cans or similar containers out of said container cleaning machine and into a container filling machine;
an arrangement configured to fill the bottles, cans or similar containers;
an arrangement configured to convey the bottles, cans or similar containers out of said container filling machine and into a container closing machine;
an arrangement configured to close the filled bottles, cans or similar containers; and
an arrangement configured to convey the closed bottles, cans or similar containers out of said container closing machine;
said arrangement configured to activate said electromagnetically activatable treatment medium in the bottles, cans or similar containers comprising an arrangement configured to activate said electromagnetically activatable treatment medium in the bottles, cans or similar containers with electromagnetic radiation in the microwave range; and
said arrangement configured to dispose said electromagnetically activatable treatment medium into an interior portion of the bottles, cans or similar containers comprises all of a), b), c) and d):
a) an arrangement configured to dispose a treatment medium comprising a mixture of at least two treatment media into an interior portion of the bottles, cans or similar containers;
b) an arrangement configured to dispose a treatment medium comprising at least one vapor component into an interior portion of the bottles, cans or similar containers;
c) an arrangement configured to dispose a treatment medium comprising hydrogen peroxide into an interior portion of the bottles, cans or similar containers; and
d) an arrangement configured to dispose a treatment medium comprising water into an interior portion of the bottles, cans or similar containers.

14. An apparatus configured to operate according to the method of claim 5, said apparatus comprising:
an arrangement configured to convey bottles, cans or similar containers into a container cleaning machine;
an arrangement configured to clean an interior portion of the bottles, said arrangement configured to clean comprising:
a rotatable rotor;
a plurality of holding elements disposed proximate a periphery of said rotor configured to hold said bottles, cans or similar containers;
an arrangement configured to dispose an electromagnetically activatable treatment medium into an interior portion of the bottles, cans or similar containers; and
an arrangement configured to activate said electromagnetically activatable treatment medium in the bottles, cans or similar containers with an electromagnetic activation source while minimizing deformation of the bottles, cans or similar containers comprising:
a Roentgen generator configured to generate electromagnetic radiation;
a Roentgen wave guide disposed about a portion of said rotor configured to activate said electromagnetically activatable treatment medium in the bottles, cans or similar containers with said electromagnetic radiation; and
a controller configured to control said magnetron;
an arrangement configured to convey the bottles, cans or similar containers out of said container cleaning machine and into a container filling machine;
an arrangement configured to fill the bottles, cans or similar containers;
an arrangement configured to convey the bottles, cans or similar containers out of said container filling machine and into a container closing machine;
an arrangement configured to close the filled bottles, cans or similar containers; and
an arrangement configured to convey the closed bottles, cans or similar containers out of said container closing machine;
said arrangement configured to activate said electromagnetically activatable treatment medium in the bottles, cans or similar containers comprising an arrangement configured to activate said electromagnetically activatable treatment medium in the bottles, cans or similar containers with electromagnetic radiation in the Roentgen range; and
said arrangement configured to dispose said electromagnetically activatable treatment medium into an interior portion of the bottles, cans or similar containers comprising all of a), b), c) and d):
a) an arrangement configured to dispose a treatment medium comprising a mixture of at least two treatment media into an interior portion of the bottles, cans or similar containers;
b) an arrangement configured to dispose a treatment medium comprising at least one vapor component into an interior portion of the bottles, cans or similar containers;
c) an arrangement configured to dispose a treatment medium comprising hydrogen peroxide into an interior portion of the bottles, cans or similar containers; and
d) an arrangement configured to dispose a treatment medium comprising water into an interior portion of the bottles, cans or similar containers.

15. The method of filling bottles, cans or similar containers of claim 5, wherein said step of activating said electromagnetically activatable treatment medium in the bottles, cans or similar containers with an electromagnetic activation source while minimizing deformation of the bottles, cans or similar containers is performed by an an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency.

16. The method of filling bottles, cans or similar containers of claim 6, wherein said step of activating said electromagnetically activatable treatment medium in the bottles, cans or similar containers with an electromagnetic activation source while minimizing deformation of the bottles, cans or similar containers is performed by an an activator configured to activate said electromagnetically activatable treatment medium in the bottles, cans or similar containers with electromagnetic radiation configured in that the frequency spectrum of the electromagnetic radiation is selected so that at least one frequency thereof is proximate to the resonance frequency of at least one component of said treatment medium or a multiple or a fraction of this resonance frequency.

17. The method of filling bottles, cans or similar containers of claim 5, wherein said container cleaning machine comprises:
a rotatable rotor;
a plurality of holding elements disposed proximate a periphery of said rotor configured to hold said bottles, cans or similar containers;
at least one dispensing arrangement being configured and disposed to dispense a treatment material into an interior portion of said bottles, cans or similar containers through an opening therein;
a magnetron configured to generate electromagnetic radiation;
a wave guide disposed about a portion of said rotor configured to activate said said electromagnetically activatable treatment medium in the bottles, cans or similar containers with said electromagnetic radiation; and
a controller configured to control said magnetron.

18. A means for filling bottles, cans or similar containers comprising:
means for cleaning an interior portion of bottles, cans or similar containers;
said means for cleaning an interior portion of bottles, cans or similar containers comprising:
means for disposing an electromagnetically activatable treatment medium into an interior portion of the bottles, cans or similar containers;
means for activating said electromagnetically activatable treatment medium in the bottles, cans, or similar containers with an electromagnetic activation source while minimizing deformation of the bottles, cans or similar containers;
means for conveying bottles, cans or similar containers into said means for cleaning an interior portion of the bottles, cans or similar containers;
means for filling bottles, cans or similar containers;
means for conveying bottles, cans or similar containers out of said means for cleaning an interior portion of bottles, cans or similar containers and into said means for filling bottles, cans or similar containers;
means for closing bottles, cans or similar containers;
means for conveying filled bottles, cans or similar containers out of said means for filling bottles, cans or similar containers and into said means for closing bottles, cans or similar containers;
means for conveying closed bottles, cans or similar containers out of said means for closing bottles, cans or similar containers.

19. The means for filling bottles, cans or similar containers of claim 18, wherein:
said means for activating said electromagnetically activatable treatment medium in the bottles, cans, or similar containers with an electromagnetic activation source while minimizing deformation of the bottles, cans or similar containers comprises means for activating said electromagnetically activatable treatment medium in the bottles, cans or similar containers with electromagnetic radiation in the microwave range; and
said means for disposing an electromagnetically activatable treatment medium into an interior portion of the bottles, cans or similar containers comprises all of a), b), c) and d):
a) means for disposing a treatment medium comprising a mixture of at least two treatment media into an interior portion of the bottles, cans or similar containers;
b) means for disposing a treatment medium comprising at least one vapor component into an interior portion of the bottles, cans or similar containers;
c) means for disposing a treatment medium comprising hydrogen peroxide into an interior portion of the bottles, cans or similar containers; and
d) means for disposing a treatment medium comprising water into an interior portion of the bottles, cans or similar containers.

20. An arrangement configured to fill bottles, cans, or similar containers according to claim 18, wherein:
said means for activating said electromagnetically activatable treatment medium in the bottles, cans, or similar containers with an electromagnetic activation source while minimizing deformation of the bottles, cans or similar containers comprises means for activating said electromagnetically activatable treatment medium in the bottles, cans or similar containers with electromagnetic radiation in the Roentgen range; and
said means for disposing an electromagnetically activatable treatment medium into an interior portion of the bottles, cans or similar containers comprises all of a), b), c) and d):
a) means for disposing a treatment medium comprising a mixture of at least two treatment media into an interior portion of the bottles, cans or similar containers;
b) means for disposing a treatment medium comprising at least one vapor component into an interior portion of the bottles, cans or similar containers;
c) means for disposing a treatment medium comprising hydrogen peroxide into an interior portion of the bottles, cans or similar containers; and
d) means for disposing a treatment medium comprising water into an interior portion of the bottles, cans or similar containers.
